(19) United States
(12) Patent Application Publication
(10) Pub. No.: US 2025/0197320 A1
(43) Pub. Date: Jun. 19, 2025

(54) STATIC MIXERS IN THE PREPARATION OF ALICYCLIC CARBOXYLIC ACIDS AND ESTERS THEREOF

(71) Applicant: Evonik Oxeno GmbH & Co. KG, Marl (DE)

(72) Inventors: Grzegorz Ziomek, Recklinghausen (DE); Thomas Schneider, Schermbeck (DE); Michael Grass, Haltern am See (DE); Berno Simon, Marl (DE)

(73) Assignee: Evonik Oxeno GmbH & Co. KG, Marl (DE)

(21) Appl. No.: 18/976,545

(22) Filed: Dec. 11, 2024

(30) Foreign Application Priority Data
Dec. 18, 2023 (EP) 23217548.9

(51) Int. Cl. C07C 5/10; B01F 25/40

(52) U.S. Cl. CPC C07C 5/10; B01F 25/40

(57) ABSTRACT

A ring hydrogenation of aromatic compounds prepares alicyclic compounds in a process for preparing alicyclic compounds using a static mixer, and also apparatuses for carrying out this process.

### CROSS-REFERENCE TO RELATED APPLICATIONS

This patent application claims priority to European Patent Application No. 23217548.9, filed on Dec. 18, 2023, in the European Patent Office, the entire disclosure of which is hereby incorporated by reference herein.

### BACKGROUND OF THE INVENTION

### Field of the Invention

The invention is in the field of the preparation of alicyclic compounds by ring hydrogenation of aromatic compounds. The invention provides processes for preparing alicyclic compounds, preferably alicyclic carboxylic acids and esters thereof, using a static mixer, and also apparatuses for carrying out this process.

Alicyclic polycarboxylic esters, such as for example the esters of cyclohexane-1,2-dicarboxylic acid, are used as lubricating oil component and as aids in metal processing. They are also used as plasticizers for polyolefins and for PVC.

For the plasticizing of PVC, esters of phthalic acid, such as for example dinonyl or didecyl esters, are predominantly used. The use of these phthalates is becoming increasingly controversial with the public and their use in plastics might be restricted. Alicyclic polycarboxylic esters, of which some have already been described in the literature as plasticizers for plastics, can be a suitable choice for possible substitutes for the plasticizers that are subject to restrictions.

### Description of Related Art

In most cases, the most economical route for the preparation of alicyclic polycarboxylic esters is the ring hydrogenation of the corresponding aromatic polycarboxylic esters, for example the above-mentioned phthalates. For this, a number of processes are already known:

U.S. Pat. No. 3,027,398 discloses the hydrogenation of dimethyl terephthalate at 110° C. to 140° C. and 35 to 105 bar over supported ruthenium catalysts.

In DE 28 23 165, aromatic carboxylic esters are hydrogenated at 70° C. to 250° C. and 30 to 200 bar over supported Ni, Ru, Rh and/or Pd catalysts to give the corresponding alicyclic carboxylic esters.

WO 99/32427 and WO 00/78704 disclose processes for hydrogenating benzene polycarboxylic esters to give the corresponding alicyclic compounds.

The hydrogenation of aromatic compounds takes place for example by reaction of an aromatics-containing starting material in the liquid phase with a hydrogen-containing hydrogenation gas. The hydrogenation gas must be dissolved in the liquid phase for this.

When introducing the hydrogenation gas and the liquid starting material into a hydrogenation unit, the concentration of hydrogen that has passed from the gas phase into the liquid phase increases over the length of the hydrogenation unit. This results in different reaction rates within the hydrogenation unit and accordingly different space-time yields in the hydrogenation unit. In the overall context, this concentration gradient therefore results in a reduced overall yield of a hydrogenation process.

### SUMMARY OF THE INVENTION

The primary object of the present invention was therefore that of providing a process for preparing alicyclic compounds, preferably alicyclic carboxylic acids and esters thereof, which enables an approximately constant space-time yield over the entire length of the hydrogenation unit.

### BRIEF DESCRIPTION OF THE DRAWINGS

FIG. 1 shows an apparatus for preparing one or more alicyclic compounds.

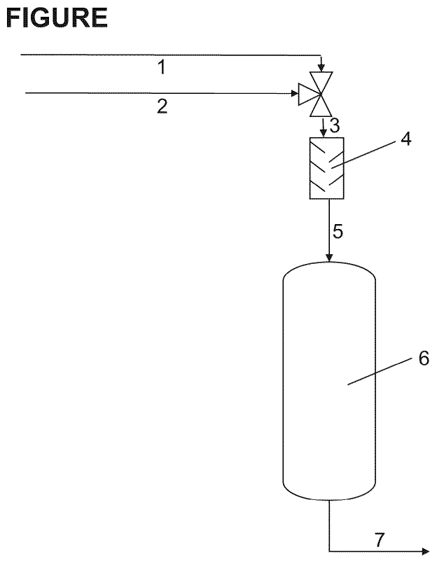

### DETAILED DESCRIPTION OF THE INVENTION

This primary object of the present invention has been achieved by providing a process for preparing one or more alicyclic compounds, comprising the steps of:
i. providing a stream A of one or more aromatic compounds and a stream B of a hydrogen-containing hydrogenation gas;
ii. bringing the streams A and B provided in step i. into contact by means of at least one to at most 8 static mixers (4), obtaining a mixed stream C (5), and introducing stream C (5) into a hydrogenation unit (6);
iii. hydrogenating the one or more aromatic compounds to give one or more corresponding alicyclic compounds in the hydrogenation unit (6);
iv. obtaining a product mixture comprising one or more alicyclic compounds.

A static mixer is a mixing element which by virtue of its internal geometry is capable of mixing two or more input streams with each other. A static mixer comprises a pipe which has flow elements and/or internals. The mixing is done by conducting the input streams over the flow elements in the pipe and turbulent flows can thus be formed. Static mixers are capable of mixing substance systems composed of two or more components having gaseous, liquid, supercritical or particulate phases.

In the present case, at least one static mixer is used. Up to 8 static mixers which are connected in parallel or in series may also be used. The use of a number of static mixers which is as low as possible is already advantageous for cost reasons. A static mixer comprises a pipe into which the two streams A and B are fed via a common inlet. The pipe has at least one and preferably two or more flow elements arranged in it. The flow element(s) effect the desired mixing of the two streams A and B to form stream C.

In the context of the present invention, it has been found that the incorporation of a static mixer into a hydrogenation unit results in mixing of the starting materials before entry into the hydrogenation unit and thus greatly increasing the concentration of hydrogen in the liquid phase immediately at the outset. This results in an improved reaction in the initial region of the hydrogenation unit.

In the context of the present invention, the term “alicyclic compounds” are to be understood as meaning those compounds which have a saturated ring system having an aliphatic structure. Such compounds are also known as cycloaliphatic compounds. Preferably, the alicyclic compounds that are obtained as products in the context of the present invention have a cyclohexane ring.

In the context of the present invention, “aromatic compounds” are to be understood as meaning those compounds which have at least one ring system which in accordance with Hückel's rule contains a number of 4n+2 delocalized electrons in conjugated double bonds, free electron pairs or unoccupied p-orbitals. Preferably, the aromatic compounds that are used as starting materials in the context of the present invention have a benzene ring.

A “hydrogen-containing hydrogenation gas” is a gas containing hydrogen. In the context of the reaction underlying the present invention, hydrogen is used as a further starting material in addition to the aromatic compounds. In the hydrogenation reaction carried out, the double bonds in the ring of the aromatic compounds used, preferably of the benzene ring, are hydrogenated by an addition reaction of the hydrogen and thereby broken. The reaction takes place in the presence of a solid catalyst. The hydrogen molecule in the hydrogen-containing hydrogenation gas intermediately bonds to the metal atom of the catalyst and the bond between the two hydrogen atoms in the hydrogen molecule is weakened and can interact with an electron-rich multiple bond (double bond). The hydrogenation takes place when two hydrogen atoms are formally in each case transferred to a double bond. This breaks the double bonds in the aromatic compounds and alicyclic compounds are obtained.

The hydrogenation gases used can be any hydrogen-containing gas mixtures which do not contain any harmful amounts of catalyst poisons such as carbon monoxide or hydrogen sulfide. The use of inert gases is optional, and preference is given to using hydrogen in a purity of greater than 95%, in particular greater than 98%. Inert gas components may for example be nitrogen or methane. It is preferable for such an amount of hydrogen to be present in the hydrogenation units that this hydrogen is present in excess, in particular in an excess of 1% to 200%, preferably in an excess of 3% to 100% and particularly preferably in an excess of 5% to 50%, based on the stoichiometric amount needed to achieve the desired conversion or the conversion that is possible in the hydrogenation unit. Setting a sufficient excess of hydrogen can have advantageous effects on the complete hydrogenation of the aromatic bonds.

In the context of the present invention, a “hydrogenation unit” is to be understood as meaning a hydrogenation reactor or two or more series-connected reactors or two or more parallel-connected reactors or a reactor group composed of parallel- and series-connected reactors. It is therefore to be understood as meaning a reactor or a reactor arrangement that can perform the function of a reactor in the process according to the invention.

The individual hydrogenation units can be charged with fresh hydrogen. However, in order to minimize the hydrogen consumption and the effluent losses resulting from the offgas, it is expedient to use the offgas of one hydrogenation unit as hydrogenation gas for another or the same hydrogenation unit. In addition, the offgas of one hydrogenation unit can be reused as fresh hydrogen after work up. For example, in a process that is carried out in two series-connected hydrogenation units each having a reactor, it is advantageous to feed fresh hydrogen into the first hydrogenation unit and to conduct the offgas of the first hydrogenation unit into the second hydrogenation unit. In this case, starting material and hydrogenation gas can flow through the hydrogenation units in opposite sequence or by way of example be mixed beforehand by means of a static mixer. It is advantageous in this process regime to keep the hydrogen excess, based on the stoichiometrically required amount, below 30%, in particular below 20%.

The hydrogenation according to the invention is preferably carried out in the liquid/gas mixed phase or liquid phase in two series-connected hydrogenation units. The first hydrogenation unit is operated in loop mode, that is to say a portion of the hydrogenation output from the first hydrogenation unit is conducted to the top of the first hydrogenation unit together with fresh starting material. Preferably, the re-feed is effected by introduction into the static mixer or by feeding without a static mixer. The other portion of the output from the first hydrogenation unit is hydrogenated in a second hydrogenation unit in straight pass mode. Preferably, a static mixer can also be present in the feed conduit to the second hydrogenation unit. Instead of one large hydrogenation unit in loop mode, it is also possible to use two or more smaller hydrogenation units in loop mode which are arranged in series or in parallel. Instead of one large hydrogenation unit with straight pass flow, it is likewise possible to operate two or more series- or parallel-connected hydrogenation units. Preferably, a static mixer is present in each feed conduit to the individual hydrogenation units. Preference is given to using one hydrogenation unit operated in loop mode and one hydrogenation unit operated in straight pass mode.

The hydrogenation can be conducted in the absence or preferably in the presence of a solvent. Solvents used may be any liquids that form a homogeneous solution with the starting material and product, are inert under hydrogenation conditions and can be easily separated off from the product. The solvent may also be a mixture of two or more substances and may optionally contain water. Preferably, when a solvent is present, this solvent is also mixed by means of the static mixer with the starting materials provided in step i.

For example, it is possible to use the following substances as solvent: Straight-chain or cyclic ethers such as tetrahydrofuran or dioxane and also aliphatic alcohols in which the alkyl radical has 1 to 13 carbon atoms.

Alcohols usable with preference as solvents are isopropanol, n-butanol, isobutanol, n-pentanol, 2-ethylhexanol, nonanols, industrial nonanol mixtures, decanol, industrial decanol mixtures, tridecanols.

When alcohols are used as solvent, it may be expedient to use that alcohol or that alcohol mixture that would form in the hydrolysis of the product. This would rule out by-product formation through transesterification. A further preferred solvent is the hydrogenation product itself.

The use of a solvent allows the aromatics concentration in the feed to the hydrogenation unit to be limited, as a result of which better temperature control in the hydrogenation unit can be achieved. This can minimize side reactions and accordingly bring about an increase in product yield. Preferably, the content of aromatic compounds in the feed to the hydrogenation unit is between 1% and 35% by weight, preferably between 2% and 25% by weight, based on the total amount of starting material. The desired concentration range in the case of hydrogenation units that are operated in loop mode can be adjusted via the circulation ratio (quantitative ratio of recycled hydrogenation output to starting material).

The process according to the invention will be described by way of example below using the example of the system shown in the FIGURE.

The starting materials are supplied to the static mixer via at least two pipelines (1, 2) which are optionally combined to form a single stream (3). Preferably, one of the two pipelines contains a hydrogen-containing hydrogenation gas and one contains aromatic compounds in liquid form. These are then introduced into the static mixer (4) and supplied as a homogeneous mixture via the pipeline (5) into the hydrogenation unit (6) in which the hydrogenation reaction takes place. The product stream (7) contains a mixture of alicyclic compounds as product and a remaining residual concentration of the starting material.

Preferably, the product stream (7) at the outlet of the hydrogenation unit contains less than 0.3% by weight, preferably less than 0.1% by weight, in particular less than 0.05% by mass, and particularly preferably 0.005% by mass, of aromatic compounds used as starting material.

It is preferred that the static mixer has a geometry which imparts a flow having a Reynolds number of greater than 100, preferably of greater than 200, in particular of greater than 500, particularly preferably of greater than 900, in the static mixer.

Those skilled in the art are capable of determining the Reynolds number based on the geometry of a static mixer. The general formula for the Reynolds number is known to those skilled in the art.

It is further preferred that the static mixer has a geometry which imparts mixing of the hydrogenation gas provided in step i. and of the provided aromatic compounds in a liquid phase such that the hydrogen from the hydrogen-containing hydrogenation gas is present in the liquid phase approximately in a saturation concentration at the end of the static mixer. Those skilled in the art are capable of determining the saturation concentration in a liquid phase.

In the context of the present invention, “approximately in a saturation concentration” is understood as meaning that the concentration of the hydrogen in the liquid phase is at most 15%, preferably at most 10%, particularly preferably at most 5% and very particularly preferably at most 1%, below the saturation concentration. This means, for example, that if the saturation concentration of hydrogen in the liquid phase is 1 g/L, then a concentration of 0.85 g/L hydrogen in the liquid phase also means that there is a saturation concentration.

It is therefore further preferred that the static mixer has a design selected from the group consisting of the mixer types: Kenics mixer, Sulzer SMV mixer, Sulzer SMX mixer, Fluitec CSE mixer and Ross ISG mixer, preferably a Kenics mixer design.

A Kenics mixer has a geometry that conveys the input stream through a helical mixing element radially in the direction of the pipe wall and then back towards the centre. An additional inversion of the flow direction and splitting of the stream result from the combination of alternating left- and right-twisting elements. These flow profiles mix the input streams.

It is preferred in the context of the present invention that the hydrogenation of the aromatic compounds provided in step i. is effected with the hydrogen-containing gas provided in step i. over one or more solid catalysts arranged in a fixed bed of the hydrogenation units.

It is further preferred that the solid catalyst includes at least one metal from the eighth transition group of the periodic table of the elements. Preference is given to using, as active metals, platinum, rhodium, palladium, cobalt, nickel or ruthenium or a mixture of two or more thereof, with ruthenium being used in particular as active metal.

In addition to the metals already mentioned, at least one metal from the first and/or seventh transition group of the periodic table of the elements is preferably additionally present in the catalysts. Preference is given to using rhenium and/or copper in addition to the metal from the eighth transition group of the periodic table of the elements.

The catalysts used in the context of this process are metals, as defined above, applied to a support material. The support materials used are preferably materials containing micropores (pore diameter less than 2 nm), mesopores (pore diameter of 2 to 50 nm) and macropores (pore diameter greater than 50 nm). For instance, with respect to the pore type, support materials having the following pore combinations are usable:
a) mesopores only,
b) micropores and mesopores,
c) mesopores and macropores,
d) micropores and mesopores and macropores,
e) micropores and macropores.

Preference is given to using, as support materials, activated carbon, silicon carbide, aluminium oxide, silicon oxide, aluminosilicate, titanium dioxide, zirconium dioxide, magnesium oxide and/or zinc oxide, or mixtures thereof.

Preferably used as support materials are solids that are largely inert under hydrogenation conditions. Examples of these are activated carbon, silicon carbide, silicon dioxide, titanium dioxide and/or zirconium dioxide, and mixtures of these compounds. Very particular preference is given to using titanium dioxides as support materials. Titanium dioxide occurs in three polymorphs (anatase, rutile and brookite), of which anatase and rutile are the most common. A preferred support material is Aerolyst 7711® (Evonik Operations GmbH). This support material consists to an extent of 15%-20% by mass of rutile and 80%-85% by mass of anatase. Further examples of suitable titanium dioxide support materials are those produced on the basis of titanium oxides from a sulfuric acid process. They generally contain >98% anatase.

In the process according to the invention, the hydrogenation in step iii. is carried out in the liquid phase or in the gas phase. The hydrogenation can be conducted continuously or batchwise over suspended catalysts or those arranged in piece form in a fixed bed. In the process according to the invention, preference is given to continuous hydrogenation over a catalyst in fixed bed form, in which the product/starting material phase is mainly in the liquid state under the reaction conditions.

It is preferred that the hydrogenation in step iii. is carried out at a pressure of 3 to 300 bar, preferably 15 to 200 bar, particularly preferably 50 to 150 bar.

It is further preferred that the hydrogenation in step iii. is carried out at a temperature of 50° C. to 250° C., preferably 70° C. to 200° C. Due to the exothermicity of the hydrogenation reaction, the reaction does not take place at a fixed temperature, but rather within a temperature range as described herein. The temperature of the reaction mixture thus rises as it flows through the hydrogenation unit.

It is preferred in the context of the process according to the invention that in step i. one or more aromatic carboxylic esters, preferably one or more aromatic mono-, di- and polycarboxylic esters, are provided.

In the context of the process according to the invention, aromatic compounds such as aromatic poly- and/or monocarboxylic acids or derivatives thereof, in particular the alkyl esters thereof, can be converted to give the corresponding alicyclic polycarboxylic acid compounds. Both full esters and partial esters can be hydrogenated. A full ester is understood to be a compound in which all acid groups have been esterified. Partial esters are compounds having at least one free acid group (or possibly anhydride group) and at least one ester group.

If polycarboxylic esters are used in the process according to the invention, these preferably contain 2, 3 or 4 ester functions.

It is preferred in the context of the process according to the invention that in step i. one or more benzene-, diphenyl-, naphthalene-, diphenyl oxide-, anthracenedi- or -polycarboxylic esters are provided. The alicyclic polycarboxylic acids obtained with the process according to the invention or the derivatives thereof consist of one or more C₆ rings where appropriate linked by a carbon-carbon bond or fused.

It is further preferred that in step i. one or more aromatic carboxylic esters having an alcohol component selected from the group consisting of branched or unbranched alkoxyalkyl, cycloalkyl and/or alkyl groups having 1 to 25 carbon atoms, preferably C₈-C₁₀ phthalate, C₈-C₁₀ terephthalate, C₈-C₁₀ isophthalate and C₈-C₁₀ trimellitate, particularly preferably di-2-ethylhexyl phthalate, diisononyl phthalate, di-2-ethylhexyl terephthalate, diisononyl terephthalate, di-2-ethylhexyl isophthalate, diisononyl isophthalate, tri-2-ethylhexyl trimellitate and triisononyl trimellitate, are provided.

Here, C₈ preferably means 2-ethylhexyl or n-octyl, C₉ means isononyl and C₁₀ means isodecyl or 2-propylheptyl.

The process is preferably a process for hydrogenating benzene-1,2-, -1,3- or -1,4-dicarboxylic esters, and/or benzene-1,2,3-, -1,2,4- or -1,3,5-tricarboxylic esters, that is to say the isomers of cyclohexane-1,2-, -1,3- or -1,4-dicarboxylic esters or of cyclohexane-1,2,3-, -1,3,5- or -1,2,4-tricarboxylic esters are obtained.

In the process according to the invention, the esters of the following aromatic carboxylic acids can for example be used: naphthalene-1,2-dicarboxylic acid, naphthalene-1,3-dicarboxylic acid, naphthalene-1,4-dicarboxylic acid, naphthalene-1,5-dicarboxylic acid, naphthalene-1,6-dicarboxylic acid, naphthalene-1,7-dicarboxylic acid, naphthalene-1,8-dicarboxylic acid, phthalic acid (benzene-1,2-dicarboxylic acid), isophthalic acid (benzene-1,3-dicarboxylic acid), terephthalic acid (benzene-1,4-dicarboxylic acid), benzene-1,2,3-tricarboxylic acid, benzene-1,2,4-tricarboxylic acid (trimellitic acid), benzene-1,3,5-tricarboxylic acid (trimesic acid), benzene-1,2,3,4-tetracarboxylic acid. It is also possible to use acids that are formed from the mentioned acids by substitution of one or more hydrogen atoms bonded to the aromatic ring with alkyl, cycloalkyl or alkoxyalkyl groups.

Preference is given to using alkyl, cycloalkyl and alkoxyalkyl esters for example of the abovementioned acids, where these radicals independently comprise 1 to 25, in particular 3 to 15, very particularly 8 to 13 carbon atoms, and in particular 9 carbon atoms. These radicals may be linear or branched. If a starting material has more than one ester group, these radicals may then be identical or different.

Examples of esters of an aromatic polycarboxylic acid that can be used in the process according to the invention include the following compounds: monomethyl terephthalate, dimethyl terephthalate, diethyl terephthalate, di-n-propyl terephthalate, dibutyl terephthalate, diisobutyl terephthalate, di-tert-butyl terephthalate, dipentyl terephthalate, monoglycol terephthalate, diglycol terephthalate, n-octyl terephthalate, diisooctyl terephthalate, di-2-ethylhexyl terephthalate, di-n-nonyl terephthalate, diisononyl terephthalate, di-2-propylheptyl terephthalate, di-n-decyl terephthalate, di-n-undecyl terephthalate, diisodecyl terephthalate, diisododecyl terephthalate, ditridecyl terephthalate, di-n-octadecyl terephthalate, diisooctadecyl terephthalate, di-n-eicosyl terephthalate, monocyclohexyl terephthalate; monomethyl phthalate, dimethyl phthalate, di-n-propyl phthalate, di-n-butyl phthalate, diisobutyl phthalate, di-tert-butyl phthalate, monoglycol phthalate, diglycol phthalate, di-n-octyl phthalate, diisooctyl phthalate, di-2-ethylhexyl phthalate, di-n-nonyl phthalate, diisononyl phthalate, di-n-decyl phthalate, di-2-propylheptyl phthalate, diisodecyl phthalate, di-n-undecyl phthalate, diisoundecyl phthalate, ditridecyl phthalate, di-n-octadecyl phthalate, diisooctadecyl phthalate, di-n-eicosyl phthalate, monocyclohexyl phthalate; dicyclohexyl phthalate, monomethyl isophthalate, dimethyl isophthalate, diethyl isophthalate, di-n-propyl isophthalate, di-n-butyl isophthalate, diisobutyl isophthalate, di-tert-butyl isophthalate, monoglycol isophthalate, diglycol isophthalate, di-n-octyl isophthalate, diisooctyl isophthalate, di-2-ethylhexyl isophthalate, di-n-nonyl isophthalate, diisononyl isophthalate, di-n-decyl isophthalate, diisodecyl isophthalate, di-n-undecyl isophthalate, diisododecyl isophthalate, di-n-dodecyl isophthalate, ditridecyl isophthalate, di-n-octadecyl isophthalate, diisooctadecyl isophthalate, di-n-eicosyl isophthalate, monocyclohexyl isophthalate.

The process according to the invention is also applicable in principle to benzoic acid and esters thereof. This is understood to mean not just alkyl benzoates but also benzoates of diols, such as for example glycol dibenzoate, diethylene glycol benzoate, triethylene glycol dibenzoate or propylene glycol dibenzoate. The alcohol component of the alkyl benzoates can consist of 1 to 25, preferably 8 to 13, carbon atoms and be in each case linear or branched.

On the industrial scale, aromatic esters, in particular full esters, are often preferably prepared from alcohol mixtures. Examples of appropriate alcohol mixtures include:
C₅ alcohol mixtures prepared from linear butenes by hydroformylation followed by hydrogenation; C₅ alcohol mixtures prepared from butene mixtures containing linear butenes and isobutene by hydroformylation followed by hydrogenation; C₆ alcohol mixtures prepared from a pentene or from a mixture of two or more pentenes by hydroformylation followed by hydrogenation; C₇ alcohol mixtures prepared from the trimerization of ethylene or dimerization of propylene or a hexene isomer or another mixture of hexene isomers, by hydroformylation followed by hydrogenation; C alcohol mixtures, such as 2-ethylhexanol (2 isomers), prepared by aldol condensation of n-butyraldehyde followed by hydrogenation; C₉ alcohol mixtures prepared from C₄ olefins by dimerization, hydroformylation and hydrogenation. The preparation of the C₉ alcohols may proceed from isobutene or from a mixture of linear butenes or from mixtures of linear butenes and isobutene. The C₄ olefins may be dimerized with the aid of various catalysts, for example protic acids, zeolites, organometallic nickel compounds or solid nickel-containing catalysts. The C₈ olefin mixtures may be hydroformylated with the aid of rhodium or cobalt catalysts; C₁₀ alcohol mixtures prepared from tripropylene by hydroformylation followed by hydrogenation; 2-propylheptanol (2 isomers) prepared by aldol condensation of valeraldehyde followed by hydrogenation; C₁₀ alcohol mixtures prepared from a mixture of at least two C₅ aldehydes by aldol condensation followed by hydrogenation; C₁₃ alcohol mixtures prepared from hexaethylene, tetrapropylene or tributene by hydroformylation followed by hydrogenation.

Other alcohol mixtures may be obtained by hydroformylation followed by hydrogenation from olefins or olefin mixtures which arise, for example, in Fischer-Tropsch syntheses, in dehydrogenations of hydrocarbons, in metathesis reactions, in the polygas process, or in other industrial processes.

Olefin mixtures with olefins of differing carbon numbers may also be used to prepare alcohol mixtures.

In the process according to the invention, any ester mixture prepared from aromatic polycarboxylic acids and the abovementioned alcohol mixtures may be used. According to the invention, preference is given to using esters prepared from phthalic acid or phthalic anhydride and a mixture of isomeric alcohols having 4 to 13 carbon atoms.

Preferably, the process according to the invention comprises the steps of:
i. providing one or more aromatic compounds selected from the group consisting of esters of phthalic acid, isophthalic acid, terephthalic acid and trimellitic acid, particularly preferably selected from the group consisting of di-n-butyl terephthalate, dipentyl terephthalate, di-2-ethylhexyl terephthalate, diisononyl terephthalate, dipentyl phthalate, di-2-ethylhexyl phthalate, diisononyl phthalate, dipentyl isophthalate, di-2-ethylhexyl isophthalate, diisononyl isophthalate, tripentyl trimellitate, tri-2-ethylhexyl trimellitate, triisononyl trimellitate, or mixtures thereof,
   and a hydrogen-containing hydrogenation gas;
ii. bringing the substances provided in step i. into contact by means of a static mixer and introducing them into a hydrogenation unit (6);
iii. hydrogenating the one or more aromatic compounds to give one or more corresponding alicyclic compounds in the hydrogenation unit (6);
iv. obtaining a product mixture comprising one or more alicyclic compounds selected from the group consisting of dialkyl cyclohexane-1,2-dicarboxylates, dialkyl cyclohexane-1,3-dicarboxylates, dialkyl cyclohexane-1,4-dicarboxylates and cyclohexane-1,2,4-tricarboxylates, particularly preferably selected from the group consisting of di-n-butyl cyclohexane-1,4-dicarboxylate, dipentyl cyclohexane-1,4-dicarboxylate, di-2-ethylhexyl cyclohexane-1,4-dicarboxylate, diisononyl cyclohexane-1,4-dicarboxylate, dipentyl cyclohexane-1,2-dicarboxylate, di-2-ethylhexyl cyclohexane-1,2-dicarboxylate, diisononyl cyclohexane-1,2-dicarboxylate, dipentyl cyclohexane-1,3-dicarboxylate, di-2-ethylhexyl cyclohexane-1,3-dicarboxylate, diisononyl cyclohexane-1,3-dicarboxylate, tripentyl cyclohexane-1,2,4-tricarboxylate, tri-2-ethylhexyl cyclohexane-1,2,4-tricarboxylate, triisononyl cyclohexane-1,2,4-tricarboxylate.

The product obtained depends on the starting material used. For example, diisononyl cyclohexane-1,2-dicarboxylate is obtained as product when using diisononyl phthalate as starting material. Accordingly, di-2-ethylhexyl cyclohexane-1,2-dicarboxylates are obtained from di-2-ethylhexyl phthalates.

Preferably, the process according to the invention comprises the steps of:
i. providing a starting material containing diisononyl phthalate (DINP) or di-2-ethylhexyl phthalate (DEHP) and a hydrogen-containing hydrogenation gas;
ii. bringing the substances provided in step i. into contact by means of a static mixer and introducing them into a hydrogenation unit (6);
iii. hydrogenating the one or more aromatic compounds to give the corresponding alicyclic compounds in the hydrogenation unit (6);
iv. obtaining a product mixture comprising diisononyl cyclohexane-1,2-dicarboxylate or di-2-ethylhexyl cyclohexane-1,2-dicarboxylate.

In addition, for example, diisononyl cyclohexane-1,4-dicarboxylate is obtained as product when using diisononyl terephthalate as starting material. Accordingly, di-2-ethylhexyl cyclohexane-1,4-dicarboxylates are obtained from di-2-ethylhexyl terephthalates.

Preferably, the process according to the invention comprises the steps of:
i. providing a starting material containing diisononyl terephthalate or di-2-ethylhexyl terephthalate and a hydrogen-containing hydrogenation gas;
ii. bringing the substances provided in step i. into contact by means of a static mixer and introducing them into a hydrogenation unit (6);
iii. hydrogenating the one or more aromatic compounds to give the corresponding alicyclic compounds in the hydrogenation unit (6);
iv. obtaining a product mixture comprising diisononyl cyclohexane-1,4-dicarboxylate or di-2-ethylhexyl cyclohexane-1,4-dicarboxylate.

In addition, for example, triisononyl cyclohexane-1,2,4-tricarboxylate is obtained as product when using triisononyl trimellitate as starting material. Accordingly, tri-2-ethylhexyl cyclohexane-1,2,4-tricarboxylates are obtained from tri-2-ethylhexyl trimellitates.

Preferably, the process according to the invention comprises the steps of:
i. providing a starting material containing triisononyl trimellitate (TINTM) or tri-2-ethylhexyl trimellitate (TOTM) and a hydrogen-containing hydrogenation gas;
ii. bringing the substances provided in step i. into contact by means of a static mixer and introducing them into a hydrogenation unit (6);
iii. hydrogenating the one or more aromatic compounds to give the corresponding alicyclic compounds in the hydrogenation unit (6);
iv. obtaining a product mixture comprising triisononyl cyclohexane-1,2,4-tricarboxylate or tri-2-ethylhexyl cyclohexane-1,2,4-tricarboxylate.

The process according to the invention is preferably conducted under the following conditions.

A Kenics mixer that imparts a Reynolds number of over 100 in the mixer is installed in the feed to a hydrogenation unit. In the mixer feed, the concentration of the aromatic compounds as starting material is between 5% and 30% by mass, in particular between 8% and 15% by mass. In the output stream from the hydrogenation unit, the concentration of the starting material is between 0.3% and 8% by mass, in particular between 1.5% and 4% by mass.

After introducing the output stream from the static mixer into the hydrogenation unit, the hydrogenation reaction is effected over a catalyst.

The specific liquid hourly space velocity (LHSV, litres of fresh starting material per litre of catalyst per hour) in the hydrogenation unit is 0.1 to 5 h⁻¹, in particular 0.5 to 3 h⁻¹.

The surface area loading in the hydrogenation unit is in the range from 25 to 140 m³/m²/h, in particular in the range from 50 to 90 m³/m²/h.

The average hydrogenation temperatures in the hydrogenation unit are 70 to 150° C., in particular 80 to 120° C.

The hydrogenation pressure in the hydrogenation unit is 25 to 200 bar, in particular 80 to 110 bar.

The process variants are suitable in particular for the hydrogenation of phthalic esters, especially for isononyl phthalates (as isomer mixture “diisononyl phthalate”, e.g. VESTINOL® 9 from Evonik OXENO GmbH & Co. KG).

A further aspect of the present invention is the provision of an apparatus for carrying out the process according to the invention, comprising at least one hydrogenation unit (6), a static mixer (4) and one or more feed streams (1, 2), wherein the static mixer is configured such that the feed streams A and B (1, 2) are brought into contact with each other and are then introduced into the hydrogenation unit (6) via a stream C (5).

The apparatus according to the invention preferably comprises two, three, four or more feed streams to the static mixer.

It is preferred that the hydrogenation unit (6) has one or more solid catalysts, preferably wherein the solid catalyst includes at least one metal from the eighth transition group of the periodic table of the elements, particularly preferably ruthenium. What has been stated herein for the catalysts used applies correspondingly.

It is further preferred that present in the hydrogenation unit is a mixture of aromatic compounds and corresponding alicyclic compounds, preferably a mixture of aromatic carboxylic esters and corresponding alicyclic compounds thereof having an alcohol component selected from the group consisting of branched or unbranched alkoxyalkyl, cycloalkyl and/or alkyl groups having 1 to 25 carbon atoms, preferably of C₈-C₁₀ phthalate, C₈-C₁₀ terephthalate, C₈-C₁₀ isophthalate and C₈-C₁₀ trimellitate, particularly preferably di-2-ethylhexyl phthalate, diisononyl phthalate, di-2-ethylhexyl terephthalate, diisononyl terephthalate, di-2-ethylhexyl isophthalate, diisononyl isophthalate, tri-2-ethylhexyl trimellitate and triisononyl trimellitate, diisononyl phthalate and/or didecyl phthalate and diisononyl cyclohexanedicarboxylates and/or didecyl cyclohexanedicarboxylates and the corresponding alicyclic compounds thereof.

A further aspect of the present invention is the use of a static mixer for bringing two or more feed streams into contact prior to introduction into one or more hydrogenation units, preferably for bringing aromatic compounds and hydrogen-containing hydrogenation gas into contact, preferably wherein the mixer has a Kenics mixer design.

It is preferred that the static mixer has a geometry which imparts a flow having a Reynolds number of greater than 100, preferably of greater than 200, in particular of greater than 500, particularly preferably of greater than 900, in the static mixer.

It is further preferred that the static mixer has a geometry which imparts mixing of the hydrogenation gas provided in step i. and of the provided aromatic compounds in a liquid phase such that the hydrogen from the hydrogen-containing hydrogenation gas is present in the liquid phase approximately in a saturation concentration at the end of the static mixer.

Preference in the context of the present invention is given to the use of the alicyclic polycarboxylic esters prepared according to the invention as plasticizers in plastics. Preferred plastics are PVC, homo- and copolymers based on ethylene, propylene, butadiene, vinyl acetate, glycidyl acrylate, glycidyl methacrylate, acrylates, acrylates with alkyl radicals, bonded to the oxygen atom of the ester group, of branched or unbranched alcohols having one to ten carbon atom(s), styrene, acrylonitrile or homo- or copolymers of cyclic olefins.

Examples of representatives of the above groups that may be mentioned include the following plastics: polyacrylates having identical or different alkyl radicals having 4 to 8 carbon atoms, bonded to the oxygen atom of the ester group, in particular having the n-butyl, n-hexyl, n-octyl and 2-ethylhexyl radical, and isononyl radical, polymethacrylate, polymethylmethacrylate, methyl acrylate-butyl acrylate copolymers, methyl methacrylate-butyl methacrylate copolymers, ethylene-vinyl acetate copolymers, chlorinated polyethylene, nitrile rubber, acrylonitrile-butadiene-styrene copolymers, ethylene-propylene copolymers, ethylene-propylene-diene copolymers, styrene-acrylonitrile copolymers, acrylonitrile-butadiene rubber, styrene-butadiene elastomers, methyl methacrylate-styrene-butadiene copolymers and/or nitrocellulose.

The alicyclic polycarboxylic esters prepared according to the invention can moreover be used for modifying plastics mixtures, for example the mixture of a polyolefin with a polyamide.

In addition to the abovementioned applications, the alicyclic polycarboxylic esters prepared according to the invention can be used as a lubricating oil component, as a constituent of cooling liquids and metal machining liquids. They can also be used as a component in paints, coatings, inks and adhesives.

1. A process for preparing one or more alicyclic compounds, the process comprising:
i. providing a stream A of one or more aromatic compounds and a stream B of a hydrogen-containing hydrogenation gas;
ii. bringing the streams A and B provided in i. into contact by at least one to at most 8 static mixers, obtaining a mixed stream C, and introducing stream C into a hydrogenation unit;
iii. hydrogenating the one or more aromatic compounds to give one or more corresponding alicyclic compounds in the hydrogenation unit; and
iv. obtaining a product mixture comprising one or more alicyclic compounds.

2. The process according to claim 1, further comprising:
feeding the two streams A and B into a pipe of the static mixer via a common inlet, wherein the pipe has arranged in it at least one flow element which effect a desired mixing of the two streams A and B.

3. The process according to claim 1, wherein the static mixer has a design selected from the group consisting of the mixer types: Kenics mixer, Sulzer SMV mixer, Sulzer SMX mixer, Fluitec CSE mixer and Ross ISG mixer.

4. The process according to claim 1, further comprising:
hydrogenating in iii. with the hydrogen-containing hydrogenation gas provided in i. over at least one solid catalyst arranged in a fixed bed of the hydrogenation unit.

5. The process according to claim 5, wherein the at least one catalyst includes at least one metal from the eighth transition group of the periodic table of the elements.

6. The process according to claim 1, further comprising:
carrying out the hydrogenation in iii. at a pressure of 3 to 300 bar.

7. The process according to claim 1, further comprising:
carrying out the hydrogenation in iii. at a temperature of 50° C. to 250° C., preferably 70° C. to 200° C.

8. The process according to claim 1, further comprising:
providing in i. one or more aromatic carboxylic esters.

9. The process according to claim 1, further comprising:
providing in i. one or more benzene-, diphenyl-, naphthalene-, diphenyl oxide-, anthracenedi- or polycarboxylic esters.

10. The process according to claim 1, further comprising:
providing in i. one or more aromatic carboxylic esters having an alcohol component selected from the group consisting of branched or unbranched alkoxyalkyl, cycloalkyl and alkyl groups having 1 to 25 carbon atoms.

11. The process according to claim 1, wherein the static mixer has a geometry which imparts a flow having a Reynolds number of greater than 100, in the static mixer.

12. An apparatus for carrying out a process according to claim 1, the apparatus comprising:
at least one hydrogenation unit,
a static mixer, and
one or more feed streams,
wherein the static mixer is configured that the one or more feed streams are brought into contact with each other and are then introduced into the hydrogenation unit via a stream.

13. The apparatus according to claim 12, wherein the hydrogenation unit has one or more solid catalysts.

14. The apparatus according to claim 12, wherein present in the hydrogenation unit is a mixture of aromatic compounds and corresponding alicyclic compounds.

15. A method, comprising:
bringing two or more feed streams into contact prior to introduction into one or more hydrogenation units by a static mixer.
